(19) United States
(12) Patent Application Publication
(10) Pub. No.: US 2013/0101564 A1
(43) Pub. Date: Apr. 25, 2013

(54) METHOD FOR PREPARING DERMIS TISSUE CELLS AGGREGATION AND USES THEREOF

(71) Applicant: Jinxi Chen, Wenzhou City (CN)

(72) Inventor: Jinxi Chen, Wenzhou City (CN)

(21) Appl. No.: 13/807,574

(22) PCT Filed: Jun. 30, 2010
(86) PCT No.: PCT/CN2010/074777

§ 371 Date: Dec. 28, 2012

(51) Int. Cl. A61K 35/36; A61K 45/06; C12N 5/071

(52) U.S. Cl. CPC A61K 35/36; C12N 5/0625; A61K 45/06
USPC 424/93.7; 435/374

(57) ABSTRACT

Dermis tissue cell aggregation, which comprises isolated dermis tissue cells with biological activity, is provided. The isolated dermis tissue cells are preserved in a mixed solution consisting of an isotonic saline solution for medical use, an anticoagulant, a nutrient for cells and a cell growth promoter, and a non-fluidity dermis tissue cell aggregation with adhesion is formed after several processes. A method for preparing the dermis tissue cell aggregation and the use thereof in preparing medicines for repairing scars are provided.

### BACKGROUND OF THE INVENTION

1. Field of the Invention

The present invention relates to a dermis tissue cell aggregation, a method for preparing the same, and the use of the same.

2. Description of the Related Art

Various pitting cicatrices and linear scars on face severely damage the looks of patients, affect beauty of their appearance, hereby disturb their study, job, marriage, sociality and lifeway. It is more severe that scars may cause significantly psychological trauma in patients, many of whom feel self-contemptuous and despaired due to having scars, and even suffer from personality distortions and psychopathy. Scars bring heavy load to patients, their families and the society. The harm of facial scars is much more severe than those occurred at other portions of the body.

Scars are formed by the reason that normal full-thickness skins cannot completely heal up due to various post-trauma defects, and a plurality of scars are formed by replacing the repaired wound with fibrous tissues. It is always a worldwide problem to treat scars, and currently, there is no technical solution or any country that can achieve such effect to treat facial scars to reach the normal or approach to normal. Up to now, conventional methods, such as “incising and suturing method” and “skin grafting method” are adopted to treat scars in medical. In “incising and suturing method”, both the wound edges after ablating a scar are roped and sutured together, but since the amount of skins is insufficient and the tension between the roped edges becomes much larger, the incision is liable to dehisce and to proliferate, thus the purpose of removing scars can not be achieved. In “skin grafting method”, the shortcoming therein is the color of the grafted skin differs significantly from those around, and there are scars remained around due to suture, and a larger scar will be remained in the position where the skin is taken.

Chinese Patent Application CN 101020899A discloses a method for treating scars by preparing a “dermis tissue cell aggregation” through the following steps: forming a wound surface by removing pigments from the surface of normal skins; using a special apparatus to cut the wound surface transversely and longitudinally to a depth between 0.3 and 0.5 mm, collecting dermis tissue fragments on the apparatus while cutting, immersing the collected dermis tissue fragments in a saline solution containing Heparin, and washing off blood coagulum; processing the fragments, and washing the processed fragments repeatedly to remove fatty particulates; accumulating the dermis tissue cells after repeatedly cutting and washing, thus producing a “dermis tissue cell aggregation” with adhesion. The “dermis tissue cell aggregation” prepared from the described method is used to treat scars, and can completely heal up the wound surface and finally form normal or approximately normal skins. The technical method using a “dermis tissue cell aggregation” may repair several pitting scars and several linear scars without being limited to the number of the scars. However, when the “dermis tissue cell aggregation” prepared from the method is employed to repair scars, there is a shortcoming of an undesired survival rate, and generally it needs to repeat several times of reparation to accomplish completely approaching normal skins.

### BRIEF SUMMARY OF THE INVENTION

In order to solve the above problem, it is the first objective of the invention to provide a dermis tissue cell aggregation which can be used for repairing facial scars of humans, can have high survival rate, good healed appearance, and can achieve effects of excellently repairing small area of facial pitting scars, post-trauma scars, post-trauma dust staining and post-operation scars.

It is the second objective of the invention to provide a method for preparing the dermis tissue cell aggregation.

It is the third objective of the invention to provide the use of the dermis tissue cell aggregation in preparing medicines for repairing scars.

To achieve the first objective, provided by the invention is a dermis tissue cell aggregation comprising isolated dermis tissue cells with biological activity, which are preserved in a mixed solution consisting of an isotonic saline solution for medical use, an anticoagulant, a nutrient for cells and a cell growth promoter.

As used herein, an isolated dermis tissue cell aggregation may be prepared with the method as described in Chinese Patent Application CN 101020899A.

As used herein, an isotonic saline solution for medicine use means that the saline solution are isotonic to the intracellular fluid in view of the electrolyte and osmotic pressure, and can be used for preserving the isolated tissue cells without dehydration or swelling dissolution induced in cells. For example, the isotonic saline solution for medicine use may adopt commercially available Ringer lactate solution.

As used herein, an anticoagulant means a medical agent capable of preventing blood from coagulating, which may select from at least one of Heparin injection and/or Chinese patent medicine injection. The Chinese patent medicine injection select from, for example, Xiangdan Injection, Radix Notoginseng Injection, Flos Carthami Injection, Radix Angelicae Sinensis Injection, and the like, and preferably, the anticoagulant is Heparin injection, or one or two of sterilized Chinese patent medicine injection. As another preferable example, the anticoagulant is Xiangdan Injection and Radix Notoginseng Injection. Addition of the anticoagulant can not only prevent coagulation of blood, but also can activate blood circulation to dissipate blood stasis, and facilitate survival, regeneration and propagation of cells in view from Chinese traditional medical. In one embodiment of the invention, in the mixed solution, the volume ratio of the anticoagulant to the isotonic saline solution for medicine use may be (0.2˜1):8. Also, those skilled in the art can determine appropriate amount of the agents depending on the facts such as actual situations and selected concentrations for the agents, etc.

As used herein, a cell growth promoter means an agent capable of facilitating survival and propagation of cells. Preferably, the cell growth promoter comprises epidermal growth factor. In another embodiment of the invention, the cell growth promoter adopts lyophilized powder for injection of recombined human epidermal growth factor. In the mixed solution, the amount of the cell growth promoter used may be 2˜10 mg per 500 ml of the isotonic saline solution for medicine use. Those skilled in the art can determine appropriate amount of the agents depending on the facts such as actual situations and selected concentrations of the agents, etc.

In the above the term “dermis tissue cell aggregation” according to the invention, the addition of the mixed solution may achieve the effects of diluting, rinsing, protecting, purifying and nourishing cells. Those skilled in the art may add each agent in a certain ratio and in a certain order depending on actual need. However, it may be noticed that the kinds and amounts of the added agents should not affect the biological activity of the isolated dermis tissue cells.

In the above the dermis tissue cell aggregation according to the invention, the amount of the mixed solution may be sufficient to immerse the dermis tissue cells. However, if the amount of the mixed solution is excessively high, condensing measure such as filtration may be subjected to the dermis tissue cell aggregation containing a large amount of the mixed solution without damaging the biological activity of the dermis tissue cells, so that the mixed solution is filtered out to obtain “residue”, i.e., the non-fluidity dermis tissue cell aggregation containing dermis tissue cells, which has somewhat adhesion. The dermis tissue cell aggregation with adhesion may be directly planted on the wound surface of scars, and the aggregation may survive in about 13 to 15 days, and thus replace and fill the pits of scars. Therefore, preferably, the amount of the mixed solution in the dermis tissue cell aggregation is sufficient to form an adhesive dermis tissue cell aggregation in a form of gel, and such a dermis tissue cell aggregation can be used directly without further processing. When using the dermis tissue cell aggregation, those skilled in the art can control the amount of the aggregation depending on the actual factors such as the area, quantity and depth of scars, and can vary it individually and flexibly depending on the constitution of the subject, for example, filling the wound surface planarly or lightly higher than the wound surface.

The dermis tissue cell aggregation provided according to the first objective of the invention is beneficial for survival and propagation of cells due to containing the substances such as anticoagulants, nutrients for cells and cell grow promoters. It can be seen from clinic observation that the aggregation with the above substances added therein is considerably enhanced in survival rate, propagation rate and post-heal appearance compared to that without these substances.

According to the second objective of the invention, provided is a method for preparing a dermis tissue cell aggregation, comprising:

(1) immersing isolated dermis tissue cells with biological activity in an isotonic saline solution for medicine use;

(2) to the mixture obtained from step (1), adding components of an anticoagulant, a nutrient for cells and a cell growth promoter in this order, wherein dispersing operation is performed after adding each component so as to make the component uniformly dispersed in a mixed solution to which they are added; after finishing addition, producing a dermis tissue cell aggregation with biological activity which is preserved in the mixed solution consisting of the anticoagulant, the nutrient for cells and the cell growth promoter.

The method for preparing a dermis tissue cell aggregation according to the second object of the invention may further comprise:

(3) subjecting condensing measure without damaging the biological activity of the dermis tissue cells to the mixture prepared from step (2), to obtain a non-fluidity dermis tissue cell aggregation with biological activity.

In step (1) of the preparation method according to the second object of the invention, the immersing isolated dermis tissue cells in a saline solution for medicine use may be performed for at least half an hour.

In step (3) of the method according to the second object of the invention, the condensing measure may be for example filtration.

In the method according to the second object of the invention, gradually adding and completely dispersing each component may make the anticoagulant, nutrient for cells and cell growth promoter sufficiently penetrate and blend into the isolated dermis tissue cells with activity, thus increase survival rate of cells, facilitate propagation of cells, and further achieve the purpose of increasing the successful rate for repairing scars and enhancing the post-heal appearance.

According to the third object of the invention, provided by the invention is the use of the dermis tissue cell aggregation in preparing medicines for repairing scars.

### DETAILED DESCRIPTION OF THE EMBODIMENTS

Hereinafter, the invention is further described in the manner of examples, the description and examples below should not be construed as limiting the scope of the invention.

### EXAMPLE 1

Isolated dermis tissue cells with biological activity were obtained by the method as described in Patent Application CN 101020899A, and were immersed in the Ringer lactate solution for half an hour. Then, the components of Xiangdan Injection, Radix Notoginseng Injection, Flos Carthami Injection, Radix Angelicae Sinensis Injection, 50% Glucose Injection and recombined human epidermal growth factor were sequentially added thereto, in which following addition of each of the components, homogenizing operation was performed once, and after another half an hour, another component was added. After addition of all the components were finished, the dermis tissue cell aggregation was produced. As for these components, Xiangdan Injection, Radix Notoginseng Injection, Flos Carthami Injection, Radix Angelicae Sinensis Injection and 50% Glucose Injection were commercially available products of Sichuan Shenghe Pharmaceutical Corporation, and the recombined human epidermal growth factor was the product of Chengdu Meixi Biological Technology Co., Ltd.

After filtering and condensing the prepared dermis tissue cell aggregation, what was obtained therefrom was a dermis tissue cell aggregation with adhesion in the form of gel, which might be directly used for repairing scars.

### EXAMPLE 2

The dermis tissue cell aggregation of the invention was prepared by the method according to Example 1, and comparative evaluation on the effect of repairing scars was performed between the prepared dermis tissue cell aggregation and that obtained by the method as described in Patent Application CN 101020899A, which were applied to the patients having scars respectively.

There were total 106 patients having scars, in which there were 88 females and 18 males aging from 20 to 42, and the portion for surgical treatment was face. The patients were divided into two groups randomly, with 53 patients for each group. The first group was performed scar reparation with the dermis tissue cell aggregation prepared by the method of Example 1, while the second group was performed scar reparation with the dermis tissue cell aggregation prepared by the method as described in Patent Application CN 101020899A. During repairing scars, the dermis tissue cell aggregation was planted on the wound surfaces of the patients' scars, and in 13 to 15 days the pledgets were opened to observe survival of the aggregation, and the patients were followed up for reparation state of scars for a long time.

When the dermis tissue cell aggregation prepared by the method of Example 1 was planted on the wound surface of scars of the first group of patients, after opening pledgets in 13 to 15 days, it could be seen that all the planted dermis tissue cell aggregations survived and formed a pink wound, and the original pitting portions of scars had disappeared. After recovering for one year, the planted portions formed a texture and appearance approximate to those of normal skins The rate for eliminating scars and approaching normal skins in one time was 70%, and the second reparation substantively approached normal, without any invalid case.

As for the second group of patients, when the dermis tissue cell aggregation prepared by the method as described in Patent Application CN 101020899A was planted on the wound surface of scars of the second group of patients, in 13 to 15 days, it could be seen that part of the planted dermis tissue cell aggregations survived, hardly could the scars be completely eliminated in one time, thus several times of reparation was required. The rate for repairing to approach normal skins in three times was 65%.

1. A dermis tissue cell aggregation comprising isolated dermis tissue cells with biological activity, characterized in that the isolated dermis tissue cells are preserved in a mixed solution consisting of an isotonic saline solution for medicine use, an anticoagulant, a nutrient for cells and a cell growth promoter.

2. The dermis tissue cell aggregation according to claim 1, characterized in that the isotonic saline solution for medicine use is Ringer lactate solution.

3. The dermis tissue cell aggregation according to claim 1, characterized in that the anticoagulant is at least one selected from Heparin injection, Xiangdan Injection, Radix Notoginseng Injection, Flos Carthami Injection and Radix Angelicae Sinensis Injection.

4. The dermis tissue cell aggregation according to claim 1, characterized in that the nutrient for cells is a Glucose Injection with a concentration of 20˜50% by weight.

5. The dermis tissue cell aggregation according to claim 1, characterized in that the cell growth promoter is epidermal growth factor.

6. The dermis tissue cell aggregation according to claim 1, characterized in that the mixed solution is used in an amount sufficient to immerse the dermis tissue cells.

7. A dermis tissue cell aggregation, characterized in that the dermis tissue cell aggregation is a non-fluidity dermis tissue cell aggregation containing dermis tissue cells prepared from subjecting condensing measure to the dermis tissue cell aggregation according to claim 6 without damaging the biological activity of the dermis tissue cells.

8. A method for preparing the dermis tissue cell aggregation according to claim 6, comprising:
(1) immersing isolated dermis tissue cells with biological activity in an isotonic saline solution for medicine use;
(2) to the mixture obtained from step (1), adding components of an anticoagulant, a nutrient for cells and a cell growth promoter in this order, wherein dispersing operation is performed after adding each of components so as to make the components uniformly dispersed in the mixed solution to which they are added; and after finishing addition, producing a dermis tissue cell aggregation with biological activity, which is preserved in the mixed solution consisting of the anticoagulant, the nutrient for cells and the cell growth promoter.

9. The method according to claim 8, characterized in that, in step (1), the isolated dermis tissue cells are immersed in the isotonic saline solution for medicine use for at least half an hour; in step (2), after adding one of the components, another component will be added in at least half an hour.

10. A method for preparing the dermis tissue cell aggregation according to claim 8, further comprising:
(3) subjecting condensing measure to the mixture prepared from step (2) without damaging the biological activity of the dermis tissue cells, to obtain a non-fluidity dermis tissue cell aggregation with biological activity.

11. A method comprising:
using dermis tissue cell aggregation to prepare medicine for repairing scars, wherein the dermis tissue cell aggregation comprises isolated dermis tissue cells with biological activity, characterized in that the isolated dermis tissue cells are preserved in a mixed solution consisting of an isotonic saline solution for medicine use, an anticoagulant, a nutrient for cells and a cell growth promoter.
